(19) United States
(12) Patent Application Publication
(10) Pub. No.: US 2009/0054925 A1
(43) Pub. Date: Feb. 26, 2009

(54) Safe tweezers

(76) Inventor: Yong Hoon Cho, Fullerton, CA (US)

Correspondence Address: Maria Erlinda C. Sarno, Esq., P.O. Box 1023, Artesia, CA, 90702, US

(21) Appl. No.: 11/894,412

(22) Filed: Aug. 21, 2007

(51) Int. Cl. A61B 17/30

(52) U.S. Cl. 606/210

(57) ABSTRACT

A covered tweezers, comprising a body having two identical thin sheets of metal fused or welded together at one end resulting in a pair of free opposing ends, the free opposing ends having a different type of tip for each type of tweezers. A cover is fixed to the body of the tweezers excepting a head portion of the tweezers. A cap encloses the head portion of the tweezers. The cover is non-metallic such as plastic, wood, rubber material or fabric. The cover is fixed to the body of the tweezers, thereby avoiding the need of a case to hold the tweezers.

This invention relates to a tweezers wherein the body of the pair of pincers is covered with a material having no sharp edges and the tip of the pincers covered with a cap when not in use.

### BACKGROUND

Tweezers in the market today pose a safety problem in that the tips are usually left uncovered which can accidentally scratch a skin surface or cause more severe damages especially when handled by an unattended child. Consequently, this problem has been addressed by providing a case where the entire tweezers can be placed and capped. However, since the tweezers have not been modified, the body which is made of metal, cause discomfort to the user especially during constant usage when the fingers are always pressing on the pincers, for example, when used in weaving or in plucking hair from the skin.

It is therefore an object of this invention to provide a tweezers where the body of the pincers is covered with a non-metallic material which have no sharp edges thereby making it comfortable in handling.

It is also an object of this invention to provide a cap or cover for the tips of the tweezers to prevent the tip from injuring someone or damaging an object when the tip pokes or scratches on these surfaces.

It is also a further object of this invention to provide a cover for the tweezers with an outer surface having a smoothly etched out portion for accommodating the fingers while these press on the tweezers during usage.

### SUMMARY OF THE INVENTION

A covered tweezers, comprising a body having two identical thin sheets of metal fused or welded together at one end resulting in a pair of free opposing ends, the free opposing ends having a different type of tip for each type of tweezers; a cover fixed to the body of the tweezers excepting a head portion of the tweezers; and, a cap for covering the head portion of the tweezers. The cover is non-metallic such as plastic, wood, rubber material or fabric. The cover is permanently fixed to the body of the tweezers, thereby avoiding the need of a case to hold the tweezers. In a plastic cover, this can be fixed by stakes on the cover having a flared tip resting on the inside surface of the thin sheet of metal of the body of the tweezers. A cap covers the head portion of the tweezers to thoroughly enclose the tweezers. The cap covers the body of the tweezers through a groove and ridge engagement. Other methods of engagement such as slip fitting the cap to the body of the covered tweezers will also work. The covered tweezers further comprises an etched out concave shaped surface at each outside surface of the cover. This etched out concave shaped surface also extends at each outside surface of the covered tweezers, that is, the concave shaped surface extends through a portion of the cap and the cover of the body of the tweezers.

Other embodiments of the present invention will become readily apparent to those skilled in the art from the following detailed description, wherein it shows and describes only certain embodiments of the invention by way of illustration. As will be realized, the invention is capable of other and different embodiments and its several details are capable of modification in various other respects, all without departing from the spirit and scope of the present invention. Accordingly, the drawings and detailed description are to be regarded as illustrative in nature and not as restrictive.

### BRIEF DESCRIPTION OF THE DRAWINGS

Aspects of the present invention are illustrated by way of example, and not by way of limitation, in the accompanying drawings, wherein:

FIG. 1 is a perspective view of the capped safe tweezers.

FIG. 2 is a perspective view of the safe tweezers with the cap detached.

FIG. 3 is an exploded view showing the parts of the safe tweezers.

FIG. 4 is a front perspective view of a cross section of one of the elongated thin sheets of the body of the tweezers showing a protruding stake of the cover inside a matching opening on the tweezer body.

FIG. 4A is a front perspective view of a cross section of one of the elongated thin sheets of the body of the tweezers showing the tip of the protruding stake of FIG. 4 flared on top of the inside surface of the elongated thin sheet to hold or fix the cover to the elongated thin sheet of the body of the tweezers.

FIG. 5 is a side view of the tweezers showing the stakes and how the tips of the stakes are melted by a heated jig.

FIG. 6 is a cross sectional view of the cap showing the ridge at one end of the cap.

FIG. 6A is a cross sectional view of a portion of the cap and the cover of the body of the tweezers showing the ridge engaging with the matching groove of the connector extending from the cover of the tweezers.

### DETAILED DESCRIPTION OF THE INVENTION

The detailed description represented herein is not intended to represent the only way or the only embodiment in which the claimed invention may be practiced. The description herein is provided merely as an example or examples or illustrations of the claimed invention and should not be construed as the only way or as preferred or advantageous over other embodiments or means of practicing the invention. Any tweezers which has a body fixed and covered with a non-metallic material and capped to prevent it from damaging or hurting someone is within the scope of this invention. The detailed description includes specific details to provide a thorough understanding of the claimed invention and it is apparent to those skilled in the art that the claimed invention may be practiced without these specific details.

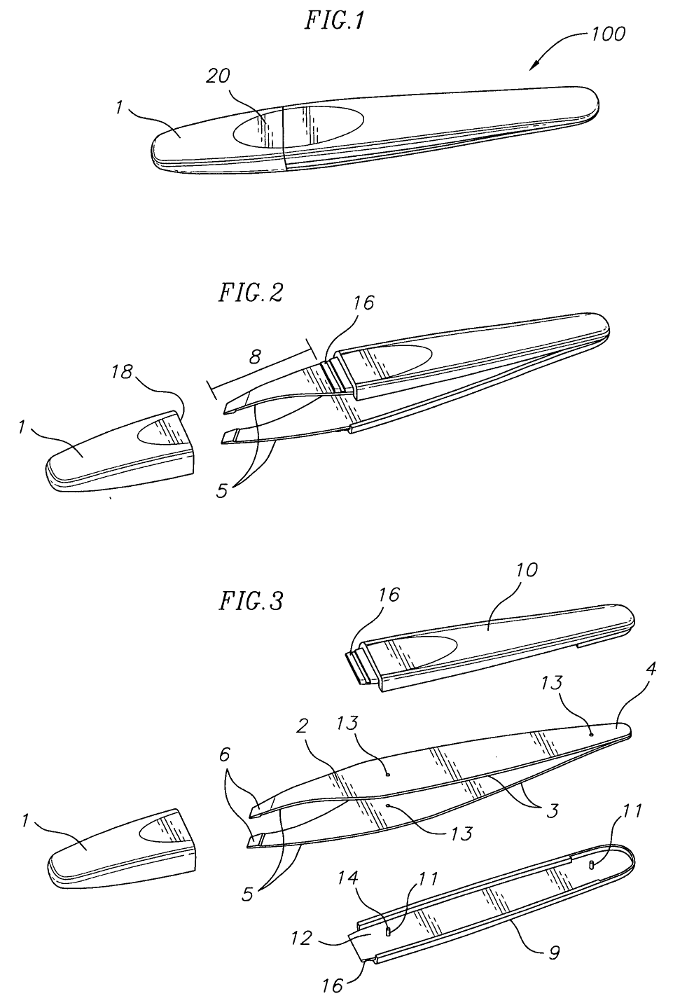

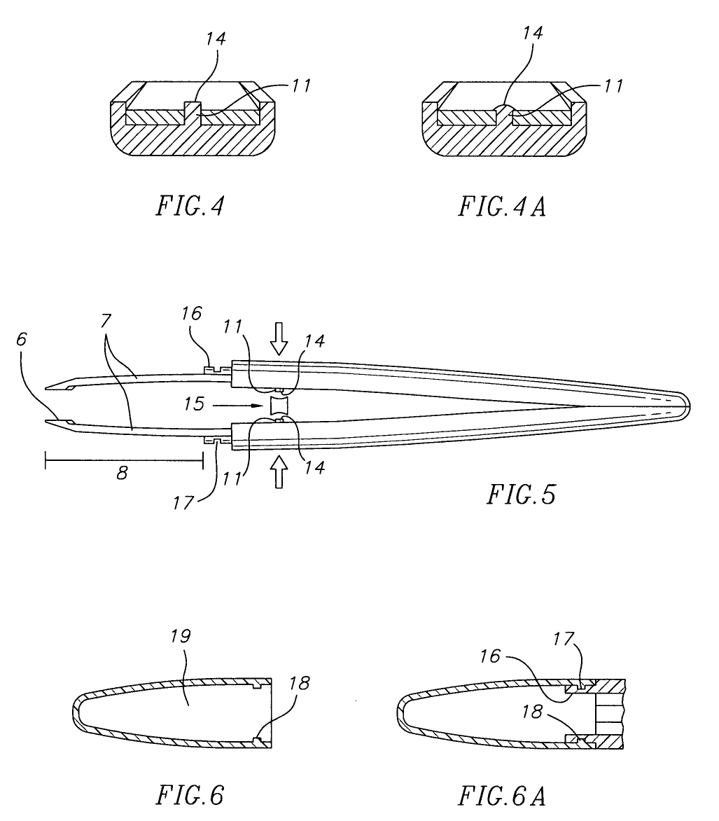

FIGS. 1 and 2 are perspective views of the safe tweezer 100, hereinafter also simply referred to as tweezers, with FIG. 1 showing the capped tweezers and FIG. 2 showing the cap 1 detached from the body 2 of the tweezers. The body 2 of an ordinary tweezers is generally constructed from two identical elongated thin sheets 3 of metal. The two elongated sheets 3 are fused or welded together at one end to form the stationary end 4 resulting in a pair of free opposing ends 5. The pair of free opposing ends opposite the fused end having the tips 6 are referred to herein as the pincer 7. The free opposing ends may be flat opening at an angle from each other or it may be curved as shown in the figures herein. The fusion at one end 4 causes the opposite end 5 to open up or space apart, consequently, allowing movement at this end as pressure by the fingers for example, is exerted or released at the outside surfaces of the elongated thin sheets 3 proximal to the free end 5. The body 2, excepting the pair of pincers with the corresponding tips 6, hereinafter also referred to as head 8, is covered with a non-metallic material to make it more comfortable to the user by preventing the metal edges of the body 2 from pressing on the fingers which can also cause callouses to develop at those areas when constantly impacted by the tweezers. FIG. 3 shows the parts of the tweezers before assembly. Both sides of body 2 are covered, one with a left cover 9 and the other with a right cover 10. These covers are made of non-metallic material such as plastic. Other non-metallic materials can be used such as synthetic and natural rubber and its derivatives, wood and fabric materials. The mode of fixing the cover to the body of the tweezers is usually dictated by the type of material used for the cover. For rubber or fabric, for example, since these are flexible and/or elastic, they can be made to wrap around the body of the tweezers. The cover can also be attached by glue or by a double sided adhesive tape. Detailed herein is a mode of attachment for plastic materials. The attachment is permanent unless force is applied to remove the cover from the body of the tweezers. Each cover has a pair of stakes or posts 11 made of the same material protruding from the interior wall 12 of the cover as shown in FIG. 3. These stakes are located at a position opposite and matching the pair of openings 13 at the elongated thin sheets 3 of the body 2 of the tweezers. To fix the cover to the body 2 of the tweezers, the stake 11 is introduced through the matching opening 13 as shown in FIG. 4 followed by pressing the tip 14 of the stake or post 11 to make the tip flare and rest on the inside surface of the elongated thin sheets 3 as shown in FIG. 4A. This operation can be automated using an ultrasonic welding machine which will press on the outside surface of the cover of the tweezers as well as melt the tip 14 of the stake by placing a is heated jig 15 between two opposing stakes as shown in FIG. 5 and pressing on the outside surface of the tweezers at the location of the stakes to flare the respective tips as shown in FIG. 4A.

The covers 9 and 10 does not extend through the entire body of the tweezers. The head 8 including the pincers with its corresponding tips 6 is left uncovered as shown in FIG. 5. At the ends of each cover proximal to the pincers are laterally protruding connectors 16 as shown in FIGS. 2, 3 and 5. The connector has a groove 17 running across the width of the connector. This groove 17 receives a corresponding ridge 18 located on the inside surface 19 of the cap 1 as shown in FIGS. 2, 6 and 6A when the cap is placed over the head 8 of the tweezers to cover the tweezers as shown in FIG. 1. FIGS. 6A shows the ridge 18 on the inside surface of the cap engaged with the groove 17 on the connector 16 extending from the cover when the tweezers is capped. The location of the ridge and groove can be reversed, the groove 17 on the inside surface of the cap and the ridge 18 on the connector 16. Other means for capping can be applied like slip fitting the cover to the connector without the use of the groove and ridge.

As shown in FIGS. 1 and 2, there is an etched concave shaped surface 20 on each outside surface of the covers to provide a better grip and comfort to the user while using the tweezers. This concave shaped surface extends to the outside surface of the cap after the tweezers is capped as shown in FIG. 1.

The tweezers can have different tips 6 on heads 8 because tweezers have found multiple usage and application. There are those used for cosmetic purposes, usually for plucking unwanted hair, there are those used in surgery for holding or gripping tissues, blood vessels, organs and the like, there are those used in dentistry for clamping a tooth, there are those used in weaving and embroidery for plucking or inserting a thread, etc.

While the embodiments of the present invention have been described, it should be understood that various changes, adaptations, and modifications may be made therein without departing from the spirit of the invention and the scope of the claims.

I claim:

1. A covered tweezers, comprising:
a body having two identical thin sheets of metal fused together at one end resulting in a pair of free opposing ends, the free opposing ends having a different type of tip for each type of tweezers;
a cover fixed to the body of the tweezers excepting a head portion of the tweezers; and,
a cap for covering the head portion of the tweezers.

2. The covered tweezers of claim 1 wherein the cover is non-metallic.

3. The covered tweezers of claim 1 wherein the cover is permanently fixed to the body of the tweezers.

4. The covered tweezers of claim 1 wherein the cover is fixed by stakes on the cover having a flared tip resting on the inside surface of the thin sheet of metal of the body of the tweezers.

5. The covered tweezers of claim 1 wherein the cap covers the head portion of the tweezers through a groove and ridge engagement.

6. The covered tweezers of claim 1 further comprising an etched concave shaped surface at each outside surface of the cover.

7. The covered tweezers of claim 1 further comprising an etched concave shaped surface extending at each outside surface of the covered tweezers.

8. A covered tweezers, comprising:
a body having two identical thin sheets of metal fused together at one end resulting in a pair of free opposing ends, the free opposing ends having a different type of tip for each type of tweezers;
a non-metallic cover permanently fixed to the body of the tweezers excepting a head portion of the tweezers; and,
a cap for covering the head portion of the tweezers.

9. The covered tweezers of claim 8 wherein the cover is fixed by stakes on the cover having a flared tip resting on the inside surface of the thin sheet of metal of the body of the tweezers,

10. The covered tweezers of claim 8 wherein the cap covers the head portion of the tweezers through a groove and ridge engagement.

11. The covered tweezers of claim 8 further comprising an etched concave shaped surface at each outside surface of the cover.

12. The covered tweezers of claim 8 further comprising an etched concave shaped surface extending at each outside surface of the covered tweezers.
